(19) United States
(12) Patent Application Publication
(10) Pub. No.: US 2006/0286596 A1
(43) Pub. Date: Dec. 21, 2006

(54) Method and apparatus for separating and purifying biopolymers

(75) Inventor: Kazuhisa Fukushima, Musashino-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: YOKOGAWA ELECTRIC CORPORATION, Musashino-shi (JP)

(21) Appl. No.: 11/507,527

(22) Filed: Aug. 22, 2006

Related U.S. Application Data
(62) Division of application No. 10/801,544, filed on Mar. 17, 2004.

(30) Foreign Application Priority Data
Apr. 8, 2003 (JP) JP 2003-103714
Apr. 8, 2003 (JP) JP 2003-103715

(51) Int. Cl. C12Q 1/68; C02F 1/469

(52) U.S. Cl. 435/6; 204/450

(57) ABSTRACT

The present invention separates and purifies a negatively charged target biopolymer from among biological samples without involving fluid movements. In other words, a first solution containing said biological samples and a second solution for preserving a separated and purified biopolymer are partitioned by a gel, thereby allowing said target biopolymer to move from within said first solution through said gel into said second solution using electrophoresis or a combination of electrophoresis and magnetophoresis so that said target biopolymer is separated and purified.

This application is a division of application Ser. No. 10/801,544 filed Mar. 17, 2004.

### BACKGROUND OF THE INVENTION

1. Field of the Invention

The present invention relates to a method and apparatus for separating and purifying target biopolymers from among biological sample biopolymers, which are used in apparatus for separating and purifying biopolymers from among biological sample cells, preprocessing units of such apparatus, cartridges for performing separation of biopolymers from cells, amplification, detection, and the like in an integrated manner, or other units.

2. Description of the Prior Art

The following descriptions exemplify DNAs as biopolymers. Methods for separating and purifying target DNAs for use in DNA chips are broadly classified into two categories: one category is based on centrifugal separation, and the other category is based on beads.

Since centrifugal separation methods require large-scale apparatus, bead-based methods are likely to become mainstream in the future as more compact systems will be preferred. An application example of a magnetic bead method, which is a technique of bead-based methods, is described in Chapter 7, “DNA Chips Employing Magnetic Beads” of “DNA Chips and It's Application” published in July 2000 by CMC Publishing Co., Ltd.

Magnetic bead methods are, for example, based on the following: Probe DNAs or probe antibodies are fixed in a certain density on the surfaces of magnetic beads; DNAs in solution are collected through complementary combination between target DNAs in solution and probes; subsequently, magnetic beads are gathered by means of magnets; after washing, DNAs are dissociated and collected from the surfaces of magnetic beads by means of using solution.

Currently, apparatus employing such magnetic bead methods, which is comparable in size to a desk-top personal computer, has become available. However, the operation of such apparatus is complicated, and a miniaturized apparatus integrated in a chip has not yet been developed.

Nevertheless, devices utilizing μTAS (micro/miniaturized total analysis system) devices have been introduced in various fields to achieve integration in chips and miniaturization. For instance, μTAS is described in Section 2, “μTAS employing micro-machine elements” of “Biochemistry, Micro Chemical Analysis System—Micro-machine Technology—” (URL:http://www.jaclap.org/LabCP/p11.html searched on Feb. 26, 2003)

In these μTAS devices, however, pumps which are drivers, valves which are controllers, mixers which are agitators, or the like have been inadequate for practical use. Consequently, only a few μTAS devices involving fluid movements have been commercialized.

This is thought to be because the dynamic characteristics of fluids change substantially at the microscopic level due to such factors as the stickiness of fluids or shapes of flow paths, and also because element technologies able to solve problems economically and functionally are still in the stage of trial and error.

Therefore, a method that is able to separate and purify target biopolymers from among biopolymers without involving fluid movements is needed.

### SUMMARY OF THE INVENTION

An object of the present invention is to solve the above-mentioned problems by providing a biopolymer separation and purification method and apparatus using the method, that is able to separate and purify target biopolymers from among biopolymers, is easy to use, could be miniaturized further, and does not involve fluid movements.

### BRIEF DESCRIPTION OF THE DRAWING

FIG. 1 illustrates a principal portion of an embodiment of apparatus for performing a biopolymer separation and purification method concerning the present invention.

FIG. 2 illustrates a principal portion of another embodiment of apparatus for performing the separation and purification method of the present invention.

FIG. 3 illustrates a principal portion of yet another embodiment of apparatus for performing the separation and purification method of the present invention.

### DETAILED DESCRIPTION OF THE PREFERRED EMBODIMENTS

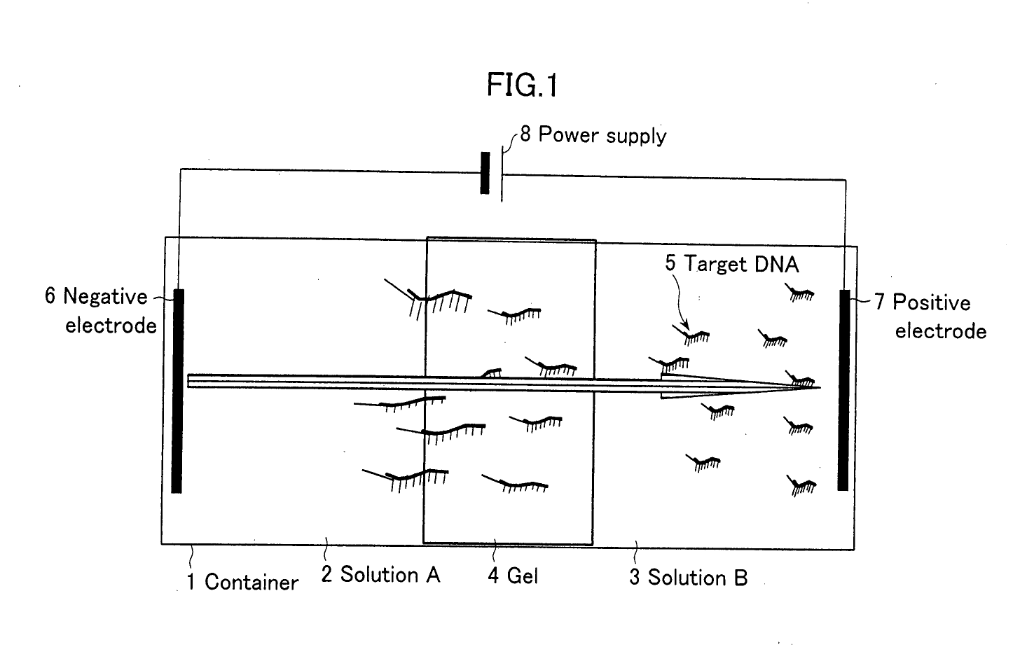

The present invention is now explained in detail with reference to the drawings. FIG. 1 illustrates a principal portion of an embodiment of apparatus for performing a biopolymer separation and purification method concerning the present invention. The present invention is intended to separate and purify negatively-charged, known target biopolymers from among biological sample biopolymers [for example, DNAs, RNAs (RNAs are transcription products from DNAs: in other words, mRNAs, rRNAs, tRNAs, or low-molecular RNAs), or proteins]. The present invention is different from a conventional method wherein an electrophoresis apparatus is used to determine or identify unknown biopolymers using electrophoresis.

In this embodiment, DNAs (more specifically, DNA fragments) are exemplified as biopolymers. In FIG. 1, a container 1 is sealed in a flat box using glass plates or the like to perform electrophoresis of DNAs. The container 1 is filled with a solution 2 (also called a solution A or a first solution) containing biological samples, a solution 3 (also called a solution B or a second solution) for preserving a separated and purified target DNA, and a gel 4 arranged between the solution A and the solution B to partition these solutions.

A negative electrode 6 and a positive electrode 7 are arranged in the solution A and the solution B respectively. Negative and positive voltages are applied from a power supply 8 to these two electrodes respectively.

Next, operations in the above-mentioned configuration are explained. Biological samples are injected into the solution A. Biological samples are a mixture of a target biopolymer (a target DNA) and other biopolymers. A target DNA 5 is separated and purified from among these biological samples in the following manner.

First, positive and negative voltages are applied from the power supply 8 to the positive electrode 7 and the negative electrode 6 respectively. Since the target DNA 5 is negatively charged, it is gravitated to the positive electrode 7 and moves from within the solution A through the gel to the solution B.

There are another types of biopolymers that are not negatively charged or whose molecules are larger (whose molecular weight is larger) than the target DNA even if they are negatively charged. Biopolymers which are not negatively charged are not gravitated to the positive electrode 7. On the other hand, biopolymers with larger molecular weights move slowly in gels and do not move with the target DNA in the solution B.

In this manner, only the target DNA 5 can be easily moved from among biological samples in the solution A into the solution B without moving the solution itself.

The present invention is not limited to the above-mentioned embodiments and includes other changes or modifications without deviating from the spirit of the present invention.

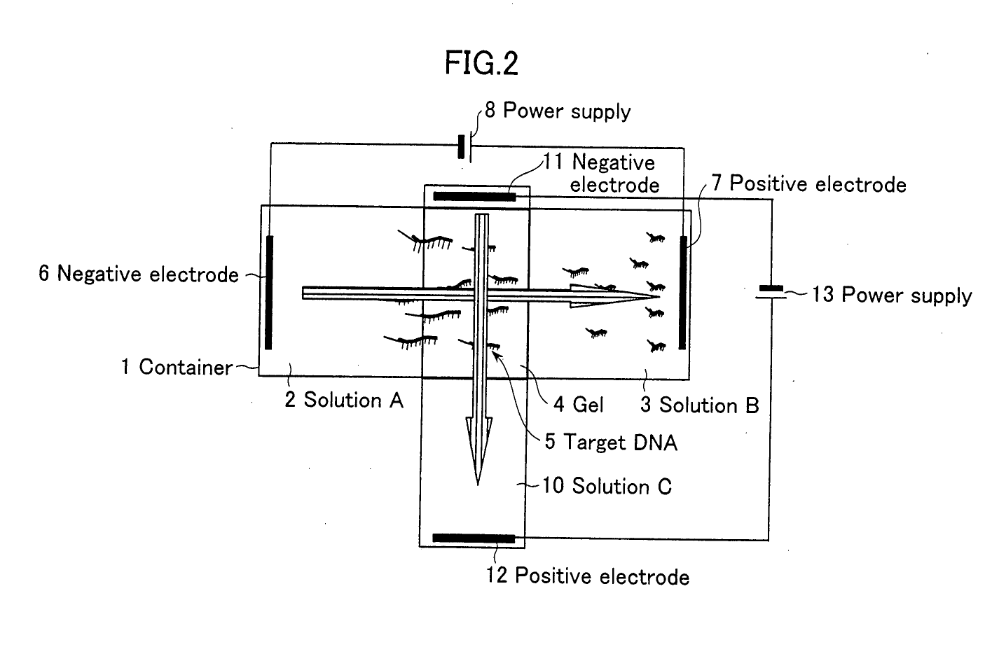

For example, if some of the biological samples in the solution A are negatively charged as in the case of the target DNA and their molecules are smaller (their molecular weights are smaller) than the target DNA, the target DNA can be easily separated and purified according to the method shown in FIG. 2.

The apparatus in FIG. 2 can also perform electrophoresis in a direction which crosses the direction of electrophoresis shown in FIG. 1 (the vertical direction in the drawing), which is explained in detail as follows:

In FIG. 2, the container 1 is formed, in addition to the configuration in FIG. 1, to be able to carry a solution 10 (also called a solution C or a third solution) which contacts the lower boundary of the gel 4. Moreover, an electrode 11 (a negative electrode) and an electrode 12 (a positive electrode) for electrophoresis are arranged at the upper boundary of the gel 4 and at the lower end portion of a third chamber respectively. Voltages can be applied from a power supply 13 to these two electrodes when necessary.

In the above-mentioned configuration, electrophoresis is continued using the power supply 8 until the target DNA is moved into the gel 4, when small molecules (molecules with small molecular weights) have already moved into the solution B.

When the target DNA 5 moves into the gel 4, application of voltage using the power supply 8 is stopped, while application of voltage using the other power supply 13 is started, thereby allowing the target DNA 5 in the gel 4 to move into the solution C. In this manner, the target DNA 5 can be easily separated from among biological samples.

In addition to the above, separation and purification operations may also be performed as follows: First, only the other DNAs with small molecules are moved into the gel 4. Then, application of voltage is switched to the power supply 13 so that biopolymers are moved into the solution C. After that, application of voltage is switched back to the power supply 8 so that the target DNA 5 is moved into the solution C through the gel 4.

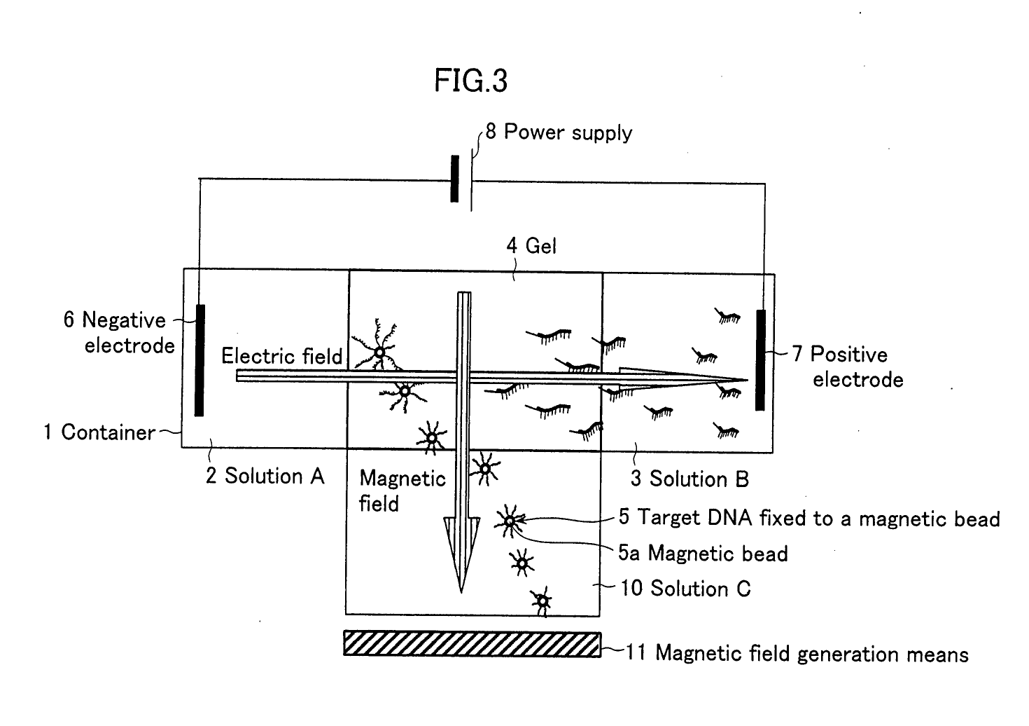

FIG. 3 illustrates a principal portion of another embodiment of the present invention. FIG. 3 is different from the configuration of FIG. 2 in that FIG. 3 has no electrodes at the upper boundary of the gel 4 and has no electrodes at the lower end portion of the third chamber; instead, a magnetic field generation means 11 is provided to generate a magnetic field and to move magnetic beads, using magnetophoresis, to the outside of the lower end of the third chamber.

Operations in this configuration are as follows: Biological samples are injected into a solution A. These samples are a mixture of a target DNA 5 fixed to a magnetic bead and other biopolymers. The target DNA is separated and purified from among these samples in the following manner: First, positive and negative voltages from a power supply 8 are applied to the positive electrode 7 and the negative electrode 6 respectively to perform electrophoresis. The negatively charged target DNA 5 and the other polymers are gravitated to the positive electrode 7 and are moved.

On the other hand, if a magnetic field is simultaneously applied in a direction towards the solution C using the magnetic field generation means 11, the target DNA 5 coupled to a magnetic bead, which is in transit in the gel 4 due to electrophoresis, is gravitated into the solution C, wherein it is separated and purified. Other biopolymers, which are in transit due to electrophoresis, are not magnetized and therefore are moved into the solution B without being affected by the magnetic field.

In the above-mentioned embodiments, a very small pillar array or a porous filter may also be used as the gel.

While DNAs were used as an example in explaining the embodiments, the present invention is not limited to DNAs and enables separation and purification of biopolymers, which are negatively charged and are coupled to magnetic beads.

In addition, an electromagnet, an electromagnetic coil, or a permanent magnet may also be used as magnetic field generation means.

As the above-mentioned explanations indicate, the present invention easily enables separation and purification of a target biopolymer from biological samples using electrophoresis or a combination of electrophoresis and magnetophoresis, without using a pump, a valve, a mixer or the like which is required in μTAS-based devices, and without involving the movement of a solution or the like.

In addition, structures or operations are sufficiently simple to easily realize a separation and purification apparatus which can also be miniaturized.

In the future, various devices based on μTAS technologies will be introduced with practical applications. On such occasions, if separation and purification of components are intended and if target components are charged, the present invention can be used for locations wherein separation and purification of such components can be performed using electrophoresis and wherein such objectives can be achieved without using a pump or a valve which make mechanisms more complicated, thus providing substantial benefits.

In addition, the present invention can be used in a section, wherein target molecules are separated and purified from among molecules or biopolymers, of a separation and purification apparatus wherein molecules or biopolymers are separated and purified from among biological cells, a preprocessing unit, a cartridge wherein the separation and purification function, the DNA amplification function and the detection reaction are performed in an integrated manner, or other units.

What is claimed is:

1. A biopolymer separation and purification apparatus, wherein a negatively charged target biopolymer is separated and purified from among biological samples, comprising:
a first solution containing said biological samples;
a second solution for preserving separated and purified biopolymers;
an electrophoresis container carrying a gel to partition said first solution from said second solution;
positive and negative electrodes provided to move said negatively charged biopolymer from within said first solution through said gel into said second solution using electrophoresis; and
a power supply for applying positive and negative voltages to said positive and negative electrodes respectively,
wherein biopolymer separation and purification can be performed by applying voltages to said electrodes and moving said target biopolymer from within said first solution through said gel to said second solution.

2. The biopolymer separation and purification apparatus of claim 1, wherein a third solution is carried in said container in order to contact said gel in a direction different from directions of said first solution and said second solution and to preserve said biopolymer moved through said gel, comprising:
positive and negative electrodes for electrophoresis which are provided to move said negatively charged biopolymer from said gel into said third solution using electrophoresis; and
a power supply for applying positive and negative voltages to said positive and negative electrodes respectively,
wherein biopolymer separation and purification can be performed by moving said target biopolymer into said second or third chamber through the switching of movement directions caused by electrophoresis.

3. The biopolymer separation and purification apparatus of claim 1, wherein a very small pillar array or a porous filter is used as said gel.

4. The biopolymer separation and purification apparatus of claim 2, wherein a very small pillar array or a porous filter is used as said gel.

5. A biopolymer separation and purification apparatus, wherein a negatively charged target biopolymer fixed to a magnetic bead is separated and purified from among biological samples, comprising:
a first solution containing said biological samples;
a second solution for preserving separated and purified biopolymers;
a third solution for preserving a separated and purified target biopolymer fixed to a magnetic bead;
a container carrying a gel to partition these three solutions from each other;
positive and negative electrodes provided in said container to move negatively charged biopolymers from within said first solution into said gel and said second solution using electrophoresis;
a power supply to apply positive and negative voltages to said positive and negative electrodes respectively; and
a magnetic field generation means wherein a magnetic field is generated in order to move said target biopolymer fixed to a magnetic bead, which is in transit in said gel using electrophoresis, into said third solution using magnetophoresis,
wherein biopolymer separation and purification can be performed by moving said target biopolymer fixed to a magnetic bead into said third solution using electrophoresis and magnetophoresis.

6. The biopolymer separation and purification apparatus of claim 5, wherein a very small pillar array or a porous filter is used as said gel.

7. The biopolymer separation and purification apparatus of claim 5, wherein an electromagnet, an electromagnetic coil, or a permanent magnet is used as said magnetic field generation means.

8. The biopolymer separation and purification apparatus of claim 6, wherein an electromagnet, an electromagnetic coil, or a permanent magnet is used as said magnetic field generation means.
